(19) United States
(12) Patent Application Publication
(10) Pub. No.: US 2016/0137603 A1
(43) Pub. Date: May 19, 2016

(54) PROCESS FOR THE PREPARATION OF CERTAIN SUBSTITUTED SULFILIMINES

(71) Applicant: Dow AgroSciences LLC, Indianapolis, IN (US)

(72) Inventors: Michael A. Gonzalez, Sanford, MI (US); Chad Meece, Attica, MI (US); Xiaoyun Chen, Midland, MI (US); Florin Dan, Midland, MI (US)

(73) Assignee: Dow AgroSciences LLC, Indianapolis, IN (US)

(21) Appl. No.: 15/000,623

(22) Filed: Jan. 19, 2016

Related U.S. Application Data
(63) Continuation of application No. 14/101,536, filed on Dec. 10, 2013.
(60) Provisional application No. 61/735,573, filed on Dec. 11, 2012.
(60) Provisional application No. 61/735,612, filed on Dec. 11, 2012.

(51) Int. Cl. C07D 213/57

(52) U.S. Cl. CPC C07D 213/57

(57) ABSTRACT

Cyano-substituted sulfilimines are produced efficiently and in high yield from the corresponding sulfides, cyanamide and hypochlorite by adding the sulfide to a solution of the cyanamide and hypochlorite in the presence of a nitrile solvent while maintaining the pH from about 8 to about 12.

### CROSS-REFERENCES TO RELATED APPLICATIONS

This application claims the benefit of U.S. patent application Ser. No. 14/101,536, which was filed on 10 Dec. 2013, which claims the benefit of, and priority from, U.S. Provisional Application Ser. No. 61/735,573, which was filed on 11 Dec. 2012, and the benefit of, and priority from, U.S. Provisional Application Ser. No. 61/735,612, which was filed on 11 Dec. 2012, the entire disclosures of these applications is hereby incorporated by reference.

### BACKGROUND OF THE INVENTION

The present invention concerns an improved process for preparing certain cyano-substituted sulfilimines.

Cyano-substituted sulfilimines are useful intermediates for the preparation of certain new insecticidal sulfoximines; see, for example, U.S. Pat. Nos. 7,678,920 B2 and 7,687,634 B2. U.S. Pat. No. 7,868,027 B2 describes the manufacture of substituted sulfilimines by the reaction of the corresponding sulfide with cyanamide and hypochlorite solution in a suitable organic solvent. While the hypochlorite process of U.S. Pat. No. 7,868,027 B2 is preferable to the iodobenzene diacetate process described in U.S. Pat. Nos. 7,678,920 B2 and 7,687,634 B2, it is plagued by significant levels of competing byproducts derived from the sulfide starting materials. It would be advantageous to produce the substituted sulfilimines efficiently and in higher yield from the corresponding sulfides by the hypochlorite route.

### SUMMARY OF THE INVENTION

Thus, the present invention concerns a process for preparing certain substituted sulfilimines, having the general structure of (I),

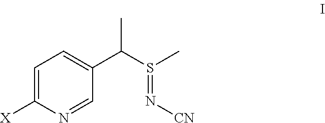

wherein X represents halogen, C₁-C₄ alkyl or C₁-C₄ haloalkyl which comprises mixing a sulfide of formula (II)

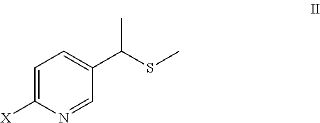

wherein X is as previously defined

with an aqueous solution of cyanamide and hypochlorite at a temperature from about −20° C. to about 10° C. in the presence of a nitrile solvent while maintaining the pH from about 8 to about 12.

### DETAILED DESCRIPTION OF THE INVENTION

Throughout this document, all temperatures are given in degrees Celsius, and all percentages are weight percentages unless otherwise stated.

The term “alkyl”, as well as derivative terms such as “haloalkyl”, as used herein, include within their scope straight chain, branched chain and cyclic moieties. Thus, typical alkyl groups are methyl, ethyl, 1-methylethyl, propyl, 1,1-dimethylethyl, and cyclopropyl. The term “haloalkyl” includes alkyl groups substituted with from one to the maximum possible number of halogen atoms, all combinations of halogens included. The term “halogen” or “halo” includes fluorine, chlorine, bromine and iodine, with fluorine being preferred.

The sulfide starting materials of Formula II or a process for their preparation have been disclosed in U.S. Pat. Nos. 7,678,920 B2 and 7,687,634 B2. The most preferred sulfide is 3-[1-(methylthio)ethyl]-6-(trifluoromethyl)pyridine (I, X=CF₃).

Cyanamide can be used as a solid or preferably as an aqueous solution. The use of a 50 weight percent solution of cyanamide in water is often preferred. A stoichiometric amount of cyanamide is required, but it is preferred to employ from about 1.5 to about 3.0 molar equivalents based on the amount of sulfide. Cyanamide also preferably should be in excess relative to hypochlorite. It is often convenient to employ from about 1.01 to about 3.0 molar equivalents of cyanamide based on the amount of hypochlorite.

By hypochlorite it is meant an aqueous solution of a metallic salt of hypochlorous acid. The metallic salt can be a Group I alkali metal salt or a Group II alkaline earth metal salt. The preferred hypochlorite salts are sodium hypochlorite or calcium hypochlorite. The aqueous hypochlorite solution usually contains from about 5 percent to about 20 percent hypochlorite salt, most preferably from about 10 percent to about 13 percent hypochlorite salt. A stoichiometric amount of hypochlorite relative to cyanamide is theoretically required but it is often preferred to employ from about 0.33 to about 0.99 molar equivalents of hypochlorite based on the amount of cyanamide. Hypochlorite also preferably should be in excess relative to sulfide. It is often convenient to employ from about 1.4 to about 2.7 molar equivalents of hypochlorite based on the amount of sulfide.

The reactions are conducted in the presence of a nitrile solvent, with acetonitrile being preferred. The nitrile solvent can be added to the aqueous mixture of hypochlorite and cyanamide prior to mixing with the sulfide, in which case the sulfide may be added neat or dissolved in additional nitrile solvent. Conversely, the sulfide dissolved in nitrile solvent may be added to an aqueous mixture of hypochlorite and cyanamide. The nitrile solvent typically comprises from about 25 wt % to about 75 wt % of the total reaction mixture.

The reactions should be performed below about 10° C. to hinder unwanted by-products formation and lower yield. While lower temperatures are beneficial, because of the presence of water in the hypochlorite and the concomitant potential for freezing and/or precipitation of salts, the most practical reaction temperature can range from about −20° C. to about 5° C. The preferred range is about −15° C. to about −5° C.

In order to minimize unwanted by-product formation and maximize yield, the hypochlorite/cyanamide mixture should be mixed with the sulfide as soon as possible after the hypochlorite/cyanamide has been mixed.

The pH is controlled from about 8 to about 12 for the hypochlorite cyanamide mixture, with about 9 to about 11 being most preferred. This can be accomplished by the addition of a base such as an aqueous solution of sodium hydroxide or by the use of a buffer such as K₃PO₄, either of which can be added prior to reaction or during reaction or both.

In addition to pH control, it is important that the sulfide be reacted with a mixture of the hypochlorite and cyanamide where cyanamide is in excess to hypochlorite. This is conveniently accomplished by premixing the hypochlorite and the cyanamide, preferably in acetonitrile, followed by mixing the resultant mixture with the sulfide, optionally also in acetonitrile. Alternately, the hypochlorite and the sulfide can be simultaneously added to the cyanamide, provided that a portion of the hypochlorite is added to the cyanamide before the addition of sulfide is commenced and this initial excess of hypochlorite is maintained throughout the simultaneous addition. The portion of hypochlorite added to the cyanamide before the addition of sulfide may range from 5-95%, with a range from 10-30% being preferred.

As appreciated by those of ordinary skill in the art, reactor design is important to achieve optimal yield. Reactors need to be designed to achieve optimal temperature control, residence time control, and mixing. Examples of potentially useful reactor designs include CSTR (continuously stirred tank reactors), plug flow reactors, and static mixers in various combinations and configurations, as well as efficient heat removal.

At the conclusion of the reaction, excess oxidants are typically reduced with NaHSO₃ or SO₂ before proceeding to the next step. The aqueous phase is separated from the organic sulfilimine phase. The organic solution of the sulfilimine can be used directly in a subsequent oxidation to an insecticidal sulfoximine or the sulfilimine can be isolated and purified by conventional techniques.

The following examples are presented to illustrate the invention.

### Examples

The experiments were conducted in Mettler Toledo EasyMax™ reactor apparatus with manual and iControl software control and data collection. The EasyMax apparatus utilized 150 milliliter (mL) glass reactor flasks equipped with electric overhead stirring (4-blade pitched down HC-22 agitator), thermowell, nitrogen pad, and Mettler Toledo dosing units (glass syringe pumps with Teflon feed tubing).

HPLC conditions are as follows:
Column: Zorbax Eclipse XDB-Phenyl 150×4.6 (5-micron); Inj V.=10 micro liter
Detector: UV at 260 nm
Flow Rate: 1.25 mL/min
Eluent: 85:15 90% water/10% MeOH to 100% acetonitrile for 15 min; 60:40 for 6 min; 85:15 for remaining 9 min (30 min total run time); or
Eluent: Reservoir A: 90% water, 10% acetonitrile, Reservoir B: 100% acetonitrile. For 15 min 85/15 A/B, then ramp over 5 min to 60/40 A/B, then at 60/40 A/B for 6 min; then ramp to 85/15 A/B over 4 min.

Qualitative Analysis:
Sample Prep: 0.2 mL reaction mixture (4-5 drops) into 1.5 mL acetonitrile/water (50:50);

Quantitative Analysis:

Sample Prep: Approx 30 mg of accurately weighed internal standard (phthalimide) was combined with approximately 200 mg of accurately weighed reaction mixture (4-5 drops) into 5.0 mL acetonitrile and 5 drops of water. 6 drops of this mixture was then added to 1.0 mL acetonitrile/water (50:50) and injected on a 5 micron loop (calibration curve/response factors generated in ChemStation software with standard grade samples of sulfilimines.

### Example 1

### Preparation of (1-{6-[trifluoromethyl]pyridin-3-yl}ethyl)-(methyl)-λ⁴-sulfanylidenecyanamide

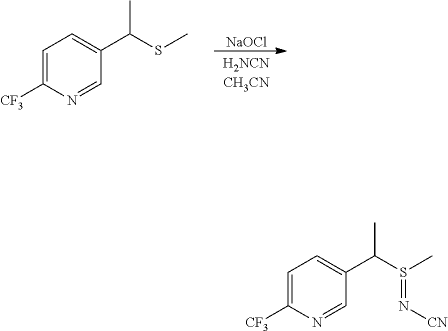

The reactor was charged with 41.0 g acetonitrile (998.8 mmol) and 1.0 g of 40% K₃PO₄ solution (2.4 mmol). Stirring was started (500 rpm) and the reactor was cooled to −5.0° C. followed by addition of 6.3 g 50% cyanamide solution (75.0 mmol), followed by initiation of a 7 hours (hr) drop-wise addition of 36.0 mL of 13% bleach (74.8 mmol hypochlorite). Approximately 10% (42 minutes (min)) into the bleach addition, 10.0 mL of 93% pure 3-[1-(methylthio)ethyl]-6-(trifluoromethyl)pyridine (51.6 mmol) was simultaneously added drop-wise over 7 hr (bleach addition was completed approximately 42 min before pyridine sulfide addition). The reaction was allowed to mix for approximately 30 min after completion of pyridine sulfide addition before warming to ambient temperature at which time the phases were separated. Both the organic and aqueous phases were analyzed by HPLC. Quantitative analysis (as described above) resulted in a weight percent of analyzed sample which was back-extrapolated using the total weight of organic phase isolated to give a 94.3% sulfilimine yield.

### Example 2

### Preparation of (1-{6-[trifluoromethyl]pyridin-3-yl}-ethyl)(methyl)-λ⁴-sulfanylidenecyanamide

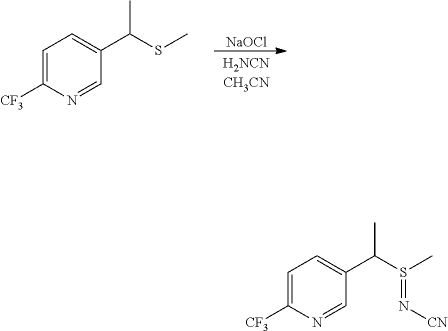

The reactor was charged with 38.0 g of bleach (66.4 mmol) and 21.0 g acetonitrile (511.6 mmol). Stirring was started (500 rpm) and the reactor was cooled to −5.0° C. followed by addition of 6.3 g of 50% cyanamide solution (75.0 mmol) over approx 1 min via pipette, which led to a rise in temperature. After the temperature returned to −5.0° C., a 6 hr drop-wise addition of 5.0 mL of 93% 3-[1-(methylthio)ethyl]-6-(trifluoromethyl)pyridine (25.8 mmol) was begun. The pH was manually controlled with a total of 1.51 g 25% NaOH (9.4 mmol) added drop wise over the 6 hr to maintain the pH in range of 10.3-10.6. The reaction was allowed to mix for approximately 30 min after completion of pyridine sulfide addition before warming to ambient temperature at which time the phases were separated. Both the organic and aqueous phases were analyzed by HPLC. Quantitative analysis (as described above) resulted in a weight percent of analyzed sample which was back-extrapolated using the total weight of organic phase isolated to give a near quantitative sulfilimine yield.

1-8. (canceled)

9. A process for preparing sulfilimines of formula (I), 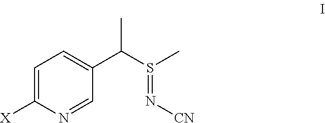
wherein X represents CF₃
said process comprising
mixing a sulfide of formula (II) 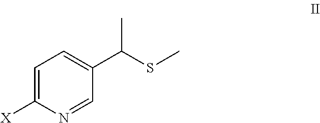
wherein X is as previously defined
with a mixture of hypochlorite, cyanamide, and acetonitrile, wherein
said cyanamide is in excess to said hypochlorite, and
1.4 to 2.7 molar equivalents of hypochlorite based on the amount of sulfide is employed, and
1.5 to 3.0 molar equivalents of cyanamide based on the amount of sulfide is employed, and
said mixing at a temperature from −20° C. to below 10° C.,
while maintaining the pH from 8 to 12.
